(19) United States
(12) Patent Application Publication
(10) Pub. No.: US 2026/0216132 A1
(43) Pub. Date: Jul. 30, 2026

(54) PRODUCT FOR ORAL ADMINISTRATION OF NUTRACEUTICALS AND PHARMACEUTICALS, AND THE PREPARATION THEREOF

(71) Applicant: Evelyn Margot-Peterman, Lexington, MA (US)

(72) Inventor: Evelyn Margot-Peterman, Lexington, MA (US)

(21) Appl. No.: 19/037,793

(22) Filed: Jan. 27, 2025

(51) Int. Cl. A61K 31/4045; A61K 9/00; A61K 9/107; A61K 31/197; A61K 31/375; A61K 33/30; A61K 35/644; A61K 47/46

(52) U.S. Cl. CPC A61K 31/4045; A61K 9/0056; A61K 9/107; A61K 31/197; A61K 31/375; A61K 33/30; A61K 35/644; A61K 47/46

(57) ABSTRACT

The present disclosure teaches a product for oral administration of nutraceuticals or pharmaceuticals, and a method for preparing the product. The product includes a frozen mixture of health-enhancing substances selected from a group consisting of nutraceuticals, pharmaceuticals, nutrients, and vegetable, herb, fungus, or fruit extracts; and a liquid base. The product may also include food additives. The method for preparing the product includes: selecting one or more health-enhancing substances from a group consisting of nutraceuticals, pharmaceuticals, nutrients, and vegetable, herb, fungus, or fruit extracts; selecting a liquid base; preparing a mixture of the one or more health-enhancing substances and the liquid base; pouring the mixture into one or more molds; freezing the one or more molds containing the mixture; demolding the mixture from the one or more molds. The method may be automated or semi-automated.

### TECHNICAL FIELD

The present disclosure relates to the field of food science and technology, pharmaceuticals and biotechnology engineering, and nutraceuticals and functional foods.

### BACKGROUND

Incorporating nutrients, nutraceuticals, pharmaceuticals, or vegetable and fruit extracts with ice may offer health benefits to certain groups of people.

First, some medical conditions may make it difficult for patients to consume liquids. Those medical conditions may include dysphagia, aspiration risk, Sjögren's Syndrome, Gastroesophageal Reflux Disease (GERD), Eosinophilic Esophagitis (EoE), esophageal cancer, neuromuscular disorders, autism, some anxiety and psychosomatic conditions, etc. Consuming ice instead of liquid may be easier for patients with those conditions for a few reasons: (1) ice melts slowly, allowing the patient to swallow small amounts of liquid at a time; (2) ice may help hydrate and soothe the throat while alleviating discomfort from dryness; (3) for some neurological conditions, ice can stimulate the nerves and muscles in the throat, potentially aiding the swallowing reflex. For patients with the aforementioned medical conditions, it could be helpful to incorporate nutrients, nutraceuticals, or pharmaceuticals with ice for consumption.

Second, consuming nutraceuticals or pharmaceuticals in the ice form may offer other health benefits. For example, many nutraceuticals and pharmaceuticals have strong or unpleasant tastes. Incorporating them into ice can make them more palatable, especially for children or individuals with taste aversions. For another example, ice-based formulations can provide a cooling effect, which may be soothing for patients with fever or heat-related illnesses. For yet another example, nutraceuticals incorporated into ice melt gradually, allowing for sustained exposure and absorption, which may be beneficial for specific nutrients, such as vitamin C, polyphenols, etc. Moreover, ice chips infused with medications may reduce oral mucositis, a common side effect of chemotherapy.

Third, adding vegetables and fruit extracts to ice may increase the attractiveness of food. For example, certain groups of people—such as children on the autism spectrum who are oversensitive to specific foods, individuals managing obesity or weight-related issues, or those with a limited inclination or habit to eat enough vegetables and fruits—may struggle to include sufficient fresh produce in their diets. These groups may benefit from consuming vegetable and fruit extract in the ice form.

Fourth, transporting, storing, and consuming vegetable and fruit extracts, as well as other nutrients, in the ice form may benefit groups of people with limited access to fresh produce, such as people living in underserved neighborhoods, rural communities, and isolated locations; those working on ships, submarines, and offshore oil rigs; and people working on scientific bases (e.g., in Antarctica) or space missions.

Fifth, some nutraceuticals and pharmaceuticals may have an unpleasant taste. Incorporating these nutraceuticals and pharmaceuticals with ice, as well as other sweeteners and food additives, may help mask the unpleasant taste by providing natural sweetness, refreshing flavors, and smooth textures. The freezing process also dulls strong or bitter tastes, making the medication more palatable while allowing it to be consumed in a more enjoyable format.

### SUMMARY

The presently disclosed technology teaches a product, comprising: a mixture, including: one or more nutraceuticals or pharmaceuticals; one or more vegetable, herb, fungus, or fruit extracts; a base that is liquid at room temperature; an emulsifier or gelling agent; wherein, the mixture is frozen in one or more molds.

In some embodiments, the base comprises electrolytes.

In some embodiments, the one or more nutraceuticals or pharmaceuticals are in liquid or powdered forms.

In some embodiments, the mixture further includes food additives.

In some embodiments, a stick, grip, or handle is inserted into each of the one or more molds.

In some embodiments, the one or more nutraceuticals or pharmaceuticals include melatonin, L-theanine, GABA, and other amino acids; the one or more vegetable, herb, fungus, or fruit extracts include chamomile extracts, Reishi mushroom extracts, saffron extracts, corydalis extracts, lion's mane mushroom extracts, and turkey tail mushroom extracts; the mixture further includes date sugar, natural flavorings, fruit juices, and essential oil; the liquid base is filtered water or comprises electrolytes.

In some embodiments, the one or more nutraceuticals or pharmaceuticals include Vitamin C, GABA, and zinc; the one or more vegetable, herb, fungus, or fruit extracts include clove extracts, chamomile extracts, Reishi mushroom extracts, saffron extracts, corydalis extracts, lion's mane mushroom extracts, turkey tail mushroom extracts, and elderberry extracts; the mixture further includes Manuka honey; the liquid base comprises electrolytes.

In some embodiments, the one or more nutraceuticals or pharmaceuticals include GABA; the one or more vegetable, herb, fungus, or fruit extracts include chamomile extracts, Reishi mushroom extracts, saffron extracts, corydalis extracts, lion's mane mushroom extracts, turkey tail mushroom extracts, and passion fruit extracts; the mixture further includes date juice; the liquid base comprises electrolytes.

In some embodiments, the one or more nutraceuticals or pharmaceuticals include Vitamin B6; the one or more vegetable, herb, fungus, or fruit extracts include fennel extracts and roman chamomile extracts; the mixture further includes natural flavor enhancers or Manuka honey; the liquid base comprises electrolytes.

In some embodiments, the one or more nutraceuticals or pharmaceuticals include Vitamin B6; the one or more vegetable, herb, fungus, or fruit extracts include ginger extracts and mint extracts; the mixture further includes protein-based additives; the liquid base comprises juice.

The present disclosure also teaches a method for preparing a product, comprising: selecting one or more nutraceuticals or pharmaceuticals, one or more vegetable, herb, fungus, or fruit extracts, and a base that is liquid at room temperature; preparing a mixture of the one or more nutraceuticals or pharmaceuticals, the one or more vegetable, herb, fungus, or fruit extracts, and the base; adding an emulsifier or gelling agent to the mixture; pouring the mixture into one or more molds; freezing the one or more molds containing the mixture; demolding the mixture from the one or more molds.

In some embodiments, the selection of the one or more nutraceuticals or pharmaceuticals, the one or more vegetable, herb, fungus, or fruit extracts, and the base is performed electronically via an I/O device, connected to a controller.

In some embodiments, a plurality of dispensers, each containing a health-enhancing substance from the one or more nutraceuticals or pharmaceuticals and the one or more vegetable, herb, fungus, or fruit extracts, is operable to automatically dispense the health-enhancing substance, based on the selection of the one or more nutraceuticals or pharmaceuticals and the one or more vegetable, herb, fungus, or fruit extracts.

In some embodiments, the plurality of dispensers is connected to a controller.

In some embodiments, the one or more molds containing the mixture are frozen in a freezer.

In some embodiments, the one or more molds containing the mixture are frozen by rapid freezing.

In some embodiments, the base includes filtered water, electrolytes, juice, milk, or plant-based milk.

In some embodiments, a stick, grip, or handle is inserted into the one or more molds.

In some embodiments, the one or more nutraceuticals or pharmaceuticals are in liquid or powdered form.

In some embodiments, the method further comprises: selecting one or more food additives; adding the one or more food additives to the mixture.

### BRIEF DESCRIPTION OF THE DRAWINGS

The present disclosure is further illustrated by way of exemplary embodiments, which are described in detail through the accompanying drawings. These embodiments are not limiting, and in these embodiments, the same numbering indicates the same structure, wherein:

FIG. 1 is a block diagram illustrating the contents of a product for oral administration of nutraceuticals and pharmaceuticals, according to some embodiments of the present disclosure.

FIG. 2 is a flow diagram illustrating a method of producing the product for oral administration of nutraceuticals and pharmaceuticals, according to some embodiments of the present disclosure.

FIG. 3 is a diagram illustrating how the method of producing the product for oral administration of nutraceuticals and pharmaceuticals may be automated for small-scale productions, according to some embodiments of the present disclosure.

### DETAILED DESCRIPTION

In order to more clearly illustrate the technical solutions of the embodiments of the present disclosure, the accompanying drawings for the description of the embodiments are described below. Obviously, the accompanying drawings in the following description are only some examples or embodiments of the present disclosure, and it is possible for a person of ordinary skill in the art to apply the present disclosure to other similar scenarios in accordance with these accompanying drawings without creative labor. Unless obviously obtained from the context or the context illustrates otherwise, the same numeral in the drawings refers to the same structure or operation.

It should be understood that the terms “system,” “device,” “unit,” and/or “module” are used herein as a way to distinguish between different components, elements, parts, sections, or assemblies at different levels. However, if other words may achieve the same purpose, the terms may be replaced with alternative expressions.

As indicated in the present disclosure and in the claims, unless the context clearly suggests an exception, the words “one,” “a,” “a kind of,” and/or “the” do not refer specifically to the singular but may also include the plural. In general, the terms “include” and “comprise” suggest only the inclusion of clearly identified steps and elements, which do not constitute an exclusive list, and the method or device may also include other steps or elements.

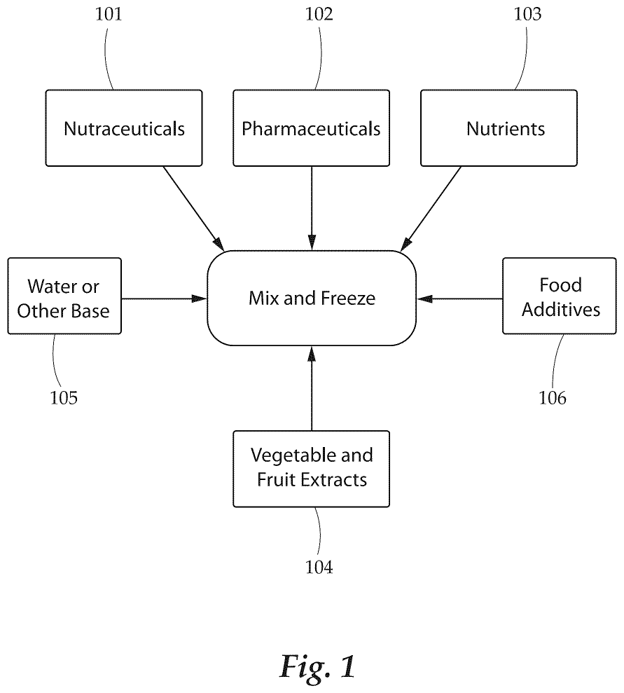

FIG. 1 is a block diagram illustrating the compositions of a product for oral administration of nutraceuticals and pharmaceuticals, according to some embodiments of the present disclosure.

As illustrated in FIG. 1, the presently disclosed product may include one or more health-promoting substances selected from the groups of nutraceuticals 101, pharmaceuticals 102, nutrients 103, vegetable, fruit, and herb extracts 104, and/or the mixture thereof. Some health-promoting substances may belong to more than one group. The weights, volumes, or proportions of the health-enhancing substances may be pre-determined. In some embodiments, the health-enhancing substances may be blended with a base 105, such as water or another liquid, to obtain a mixture. The mixture may also include one or more food additives 106. The mixture may then be molded and frozen to obtain the presently disclosed product.

In some embodiments, the presently disclosed product may include one or more nutraceuticals 101. Nutraceuticals are products derived from food sources that provide additional health benefits beyond basic nutritional value. They may help prevent or treat diseases, promote general health and immunity, or enhance physiological functions. These nutraceuticals 101 may include vitamins and minerals, such as Vitamin D, Vitamin C, zinc, magnesium, and selenium; herbal supplements, such as echinacea, elderberry, ashwagandha, ginseng, and turmeric; probiotics and prebiotics, such as lactobacillus, bifidobacterium, inulin, fructooligosaccharides (FOS). In some embodiments, the nutraceuticals 101 may improve a consumer's brain and cognitive health. These nutraceuticals 101 may include Omega-3 fatty acids, such as DHA and EPA; adaptogens, such as Rhodiola rosea extract and Hericium erinaceus extract; and nootropics, such as Bacopa extract and Ginkgo extract. In some embodiments, the nutraceuticals 101 may improve a consumer's heart health. These nutraceuticals 101 may include cardiovascular support supplements, such as coenzyme Q10 (CoQ10), red yeast rice extract, garlic extract, and policosanol; fiber supplements, such as psyllium husk and beta-glucans. In some embodiments, the nutraceuticals 101 may improve a consumer's joint and bone health. These nutraceuticals 101 may include anti-inflammatory compounds, such as glucosamine, chondroitin, and methylsulfonylmethane (MSM); collagen; and calcium and Vitamin K2. In some embodiments, the nutraceuticals 101 may improve a consumer's skin health, and/or have anti-aging effects. These nutraceuticals 101 may include antioxidants, such as resveratrol, astaxanthin, green tea extract (EGCG), and alpha-lipoic acid; hyaluronic acid; and collagen peptides. In some embodiments, the nutraceuticals 101 may provide benefits to a consumer's level of energy and metabolism. These nutraceuticals 101 may include energy boosters, such as L-carnitine, nicotinamide riboside (NR), and weight management supplements, such as garcinia cambogia and conjugated linoleic acid (CLA). The nutraceuticals 101 may also provide benefits to hormonal balance, which may include maca root extract and evening primrose extract. The nutraceuticals 101 may also improve a consumer's sleep and relaxation, which may include melatonin and L-theanine.

In some embodiments, the presently disclosed product may include one or more pharmaceuticals 102. These pharmaceuticals 102 may include pain relief and anti-inflammatory medications, such as acetaminophen, ibuprofen, and aspirin; antibiotics, such as amoxicillin, azithromycin, and ciprofloxacin; cardiovascular medications, such as atorvastatin, lisinopril, and metoprolol; mental health medication, such as sertraline, lorazepam, and lithium; medications for diabetes management, such as metformin and insulin; allergies and respiratory medication, such as loratadine and albuterol; gastrointestinal medications, such as omeprazole and loperamide; orally consumed vaccines and antivirals, such as oseltamivir and acyclovir. The pharmaceuticals 102 may be prescription or over-the-counter (OTC).

In some embodiments, the presently disclosed product may include one or more nutrients 103. The one or more nutrients 103 may include vitamins, such as Vitamin A, Vitamin D, Vitamin E, Vitamin K (including K1 and K2), Vitamin C, and B-Vitamins (B1, B2, B3, B5, B6, B7, B9, B12); minerals, such as calcium, iron, magnesium, zinc, iodine, selenium, copper, potassium, phosphorus, chromium, manganese; macronutrients, such as protein, dietary fiber, and unsaturated fats; probiotics; prebiotics; antioxidants, such as selenium, Vitamin E, and Vitamin C; electrolytes, such as sodium, potassium, magnesium, and calcium; phytochemicals, such as polyphenols and carotenoids; amino acids, such as L-glutamine and taurine; herbal extracts; choline; coenzyme Q10 (CoQ10).

In some embodiments, the presently disclosed product may include one or more vegetable, fruit, fungus, or herb extracts 104. The vegetable, fruit, fungus, or herb extracts 104 may include fruit extracts, such as citrus extracts, berry extracts, apple extracts, grape extracts, and tropical fruit extracts; vegetable extracts, such as leafy green extracts, carrot extracts, beetroot extracts, tomato extracts, and allium extracts. The vegetable, fruit, fungus, or herb extracts 104 may also include herb extracts, such as vanilla extract, mint extract, basil extract, rosemary extract, thyme extract, oregano extract, cilantro extract, parsley extract, lavender extract, lemon balm extract, sage extract, fennel extract, chamomile extract, etc. The herb extracts may offer health benefits, provide nutritious values, or function as natural flavor enhancers or sweeteners. The vegetable, fruit, fungus, or herb extracts 104 may also include fungus extracts, such as Reishi mushroom extract, lion's mane mushroom extract, turkey tail mushroom extract, etc. The vegetable, fruit, fungus, or herb extracts 104 may be produced in various ways, such as juicing and pressing, drying and grinding, solvent extraction, steam distillation, supercritical CO₂ extraction, enzymatic extraction, etc. In some embodiments, for health-conscious consumers, the vegetable, fruit, fungus, or herb extracts 104 may come from organic produces.

In some embodiments, the presently disclosed product may also include water or other liquid bases 105. In one embodiment, the presently disclosed product may use electrolyte as the base 105. Electrolyte liquids can replenish essential minerals like sodium, potassium, and magnesium, promoting hydration, muscle function, and recovery from dehydration due to exercise, heat, or illness. They can quickly restore fluid and electrolyte balance in the body. In other embodiments, other liquids can also be used as the base 105, such as fruit juice, coconut water, milk or plant-based milk, yogurt, herbal teas, coconut milk or other plant-based milk, and coffee.

In some embodiments, the presently disclosed product may also include one or more food additives 106. The food additives 106 may include flavor enhancers, such as citric acid and natural or artificial flavors; sweeteners, such as sugar, corn syrups, and stevia or erythritol; stabilizers and emulsifiers, such as guar gum or xanthan gum, or pectin; color additives, such as dyes derived from fruits and vegetables, or artificial food coloring; preservatives, such as ascorbic acid, sodium benzoate or potassium sorbate; anti-stick agents, such as vegetable glycerin; other functional additives, such as gelatin, agar-agar, or salt. In some embodiments, for health-conscious consumers, the food additives 106 may also include naturally produced flavor enhancers, like fresh herbs and spices, such as mint, basil, cinnamon, and ginger; citrus zest, such as lemon, lime, or orange zest; vanilla or almond extract; fruit purees; coconut water. Likewise, the food additives 106 may also include natural sweeteners, such as honey, maple syrup, agave nectar, dates, stevia, monk fruit sweetener, coconut sugar, ripe bananas, and molasses.

In one embodiment, the compositions of the presently disclosed product may include date sugar, chamomile extracts, natural flavorings, fruit juices, melatonin, L-theanine and other amino acids, and essential oil, with electrolyte or filtered water as the base 105. The essential oil may be lemon-based. This combination of health-enhancing substances may improve a consumer's relaxation and sleep.

In one embodiment, each piece of the presently disclosed product may include 2-6 grams of date sugar, 25-100 milligrams of chamomile extracts, trace amounts of natural flavoring, 15-40 mL of fruit juices, 0.1-2 milligrams of melatonin, 25-100 milligrams of L-theanine and other amino acids, trace amounts of essential oil, and 40-80 mL of electrolyte or filtered water.

In one embodiment, the health-enhancing substances of the presently disclosed product may include clove extracts, chamomile extracts, Manuka honey, Vitamin C, zinc, and elderberry extracts, with electrolyte as the base 105. This combination of health-enhancing substances may alleviate throat pain or soreness.

In one embodiment, each piece of the presently disclosed product may include 5-20 milligrams of clove extracts, 25-100 milligrams of chamomile extracts, 2-5 grams of Manuka honey, 15-30 milligrams of Vitamin C, 2-5 milligrams of zinc, 50-150 milligrams of elderberry extracts, and 40-80 mL of electrolyte base.

In one embodiment, the health-enhancing substances of the presently disclosed product may include chamomile extracts, passion fruit extracts, date juice, or other natural sweeteners, with electrolyte as the base 105. The chamomile extracts may be from German chamomile. This combination of health-enhancing substances may alleviate anxiety.

In one embodiment, each piece of the presently disclosed product may include 25-100 milligrams of chamomile extracts, 5-20 milligrams of passion fruit extracts, 5-10 mL of date juice, or other natural sweeteners, and 40-80 mL of electrolyte base.

In one embodiment, the health-enhancing substances of the presently disclosed product may include fennel extracts, roman chamomile extracts, Vitamin B6, and natural flavor enhancers and/or natural sweeteners, with electrolyte as the base 105. This combination of health-enhancing substances may improve a consumer's digestion.

In one embodiment, each piece of the presently disclosed product may include 10-30 milligrams of fennel extracts, 25-75 milligrams of roman chamomile extracts, 0.5-2 milligrams of Vitamin B6, trace amounts of natural flavor enhancers and/or 1-5 grams of natural sweeteners, and 40-80 mL of electrolyte base.

In one embodiment, the health-enhancing substances of the presently disclosed product may include ginger extracts, Vitamin B6, mint extracts, and protein-based additives, with juice as the base 105. This combination of health-enhancing substances may help enhance a consumer's energy level.

In one embodiment, each piece of the presently disclosed product may include 10-30 milligrams of ginger extracts, 0.5-2 milligrams of Vitamin B6, 10-30 milligrams of mint extracts, 2-6 grams of protein-based additives, and 40-80 mL of juice. In some embodiments, the protein-based additives may be one or more types of proteins that dissolve well in liquid and are also suitable for freezing. For example, the protein-based additives may be collagen peptides or hydrolyzed protein.

In the embodiments above, wherein the presently disclosed product includes chamomile extracts, other ingredients providing calming and sedative effects such as Reishi mushroom extracts, saffron extracts, corydalis extracts, lion's mane mushroom extracts, turkey tail mushroom extracts, and/or GABA (gamma-aminobutyric acid) may also be included in the presently disclosed product.

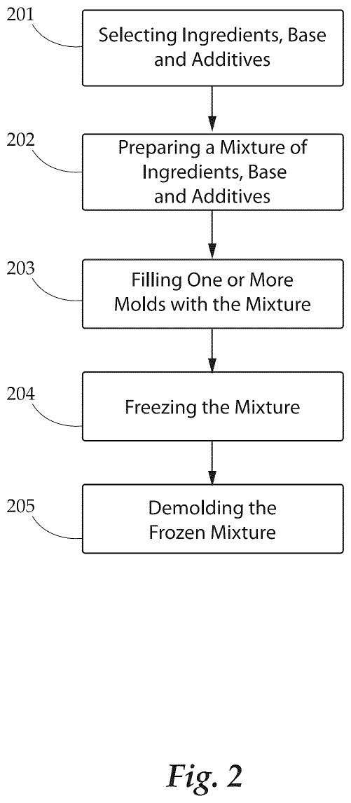

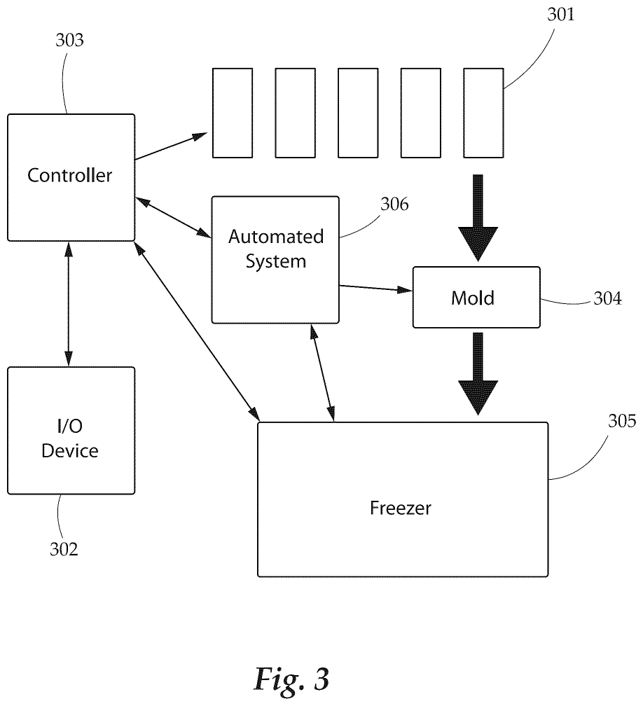

FIG. 2 is a flow diagram illustrating a method of producing the product for oral administration of nutraceuticals and pharmaceuticals, according to some embodiments of the present disclosure. The presently disclosed product may be produced either on a large scale in an industrial setting or on a small scale by a consumer or a healthcare provider. The presently disclosed method may be carried out manually, partially automatedly, or entirely automatedly. For example, FIG. 3 is a diagram illustrating how the method of producing the product for oral administration of nutraceuticals and pharmaceuticals may be automated for small-scale productions, according to some embodiments of the present disclosure.

At 201, a consumer, manufacturer, or healthcare provider may select one or more health-promoting substances from the groups of nutraceuticals 101, pharmaceuticals 102, nutrients 103, and vegetable and fruit extracts 104, as well as a base 105 and one or more food additives 106, based on the end consumers' needs and desires. In an industrial setting, the step 201 may be a managerial or engineering decision, and the presently disclosed product with the chosen health-enhancing substances, base, and food additives may be produced in large bulks. For example, a manufacturer may produce fruit and vegetable extracts in the iced form in large bulks for people with limited access to fresh produce.

The preparation of the presently disclosed product may also be on a much smaller scale. For example, a patient having trouble consuming liquid may need to take a medication in the iced form. In this case, only a small quantity of the presently disclosed product is needed. A healthcare provider of the patient may choose the medication, or the health-enhancing substances of the medication, from the group of available pharmaceuticals 102. The healthcare provider may choose water as the base 105. To cover the potentially undesirable flavor of the medication, the healthcare provider may also choose one or more natural sweeteners from the group of food additives 106.

In some embodiments, the preparation of the presently disclosed product on a small scale may be automated. For example, a machine may include a plurality of dispensers 301 containing health-enhancing substances in powdered or liquid form from the groups of nutraceuticals 101, pharmaceuticals 102, nutrients 103, and vegetable and fruit extracts 104; liquids acting as bases 105; and one or more food additives 106. A user, such as a healthcare provider, may select one or more health-enhancing substances, bases, and food additives from an I/O device 302 in communication with the dispensers 301. In some embodiments, the I/O device 302 may be a computerized device or a touchscreen. In some embodiments, the I/O device 302 may connect to the dispensers 301 via wired or wireless connections. In some embodiments, the I/O device 302 may communicate with the dispensers 301 via a controller 303. The dispensers 301 may operate according to the user's selection. In some embodiments, the dispensers 301 may be controlled by buttons or toggles, making the process semi-automated.

In some embodiments, the controller 303 may be pre-stored with a plurality of combinations of health-enhancing substances that are recommended against. For example, calcium supplements and iron supplements should not be consumed together because calcium may interfere with the absorption of iron in the intestines. For another example, Vitamin K should not be consumed together with blood thinners because Vitamin K can counteract the effects of blood-thinning medications. Alternatively, the controller 303 may be connected to the internet, and an artificial intelligence (AI) agent implemented on the controller 303 may be operable to generate the plurality of combinations of health-enhancing substances that should not be consumed together based on information available on the internet or check if a certain combination of health-enhancing substances should be recommended against. In some embodiments, if the user's selection of one or more health-enhancing substances is or includes a combination of health-enhancing substances that is recommended against, the controller 303 may send a warning to the I/O device 302.

In some embodiments, some of the nutraceuticals 101, pharmaceuticals 102, nutrients 103, and vegetable and fruit extracts 104 may be pre-processed before usage. For example, some health-enhancing substances may need to be powdered or dissolved in a liquid before usage. For another example, some vegetable or fruit extracts 104 may be produced from raw vegetables or fruits.

At 202, a mixture of the selected compositions, including the health-enhancing substances, the base, and the food additives, may be prepared. In some embodiments, the selected health-enhancing substances may be blended in a container. In some embodiments, the container may be a mold. In other embodiments, the container may not be a mold, and the mixture may be filled into one or more molds at 203. In some embodiments, a stick, a grip, or a handle may be put into each of the one or more molds for a consumer to handhold the product with ease.

If the product is produced on an industrial scale, the mixture may be prepared in a large, automated tank. Then, the prepared mixture may be poured into molds on a conveyor system. The stick, grip, or handle may also be inserted into the molds using automated machinery.

If the product is produced on a small scale, the mixture may either be manually blended in a container or automatically prepared using a countertop blender, an immersion blender, an electric whisk, an automatic mixer, etc. The prepared mixture may also be manually or automatically transferred into one or more molds. In some embodiments, the dispensers 301 may dispense the selected health-enhancing substances directly into one or more mold(s) 304, where the selected health-enhancing substances may be blended. Alternatively, the dispensers 301 may dispense the selected health-enhancing substances into an intermediated container for the blending of the selected health-enhancing substances. The mixture may then be transferred into the one or more mold(s) 304 using an automated system 306 controlled by the controller 303, wherein the automated system may consist of a number of motors, sensors, and conveyor belts. In some embodiments, the user may select the number, size, and type of mold(s) 304 using the I/O device 302, and the automated system may place the selected mold(s) accordingly. The quantity of compositions to be dispensed by the dispensers 301 may also be calculated by the controller 303 according to the user's input via the I/O device 302.

At 204, the mixture may be frozen. In an industrial setting, the molds may pass through a freezing tunnel or are immersed in a glycol or brine solution at low temperatures. In some embodiments, rapid freezing may be used. Rapid freezing may prevent large ice crystals, ensuring a smooth texture. For small-scale productions, the mold(s) may be transferred into a freezer 305, either manually or using the automated system 306. In some embodiments, the temperature and other settings of the freezer 305 may be controlled and monitored by the controller 303, via the automated system. In some embodiments, the user may adjust the temperature of the freezer 305 and/or the length of freezing time via the I/O device 302. In some embodiments, the freezing may take 4-6 hours. In certain embodiments, the molds may be frozen in a flash freezer at cryogenic temperatures. This rapid freezing prevents settling out or separation of important components, helping to ensure uniform or approximately uniform consistency. Examples of flash freezing temperatures may include, but are not limited to temperatures below −10 degrees Fahrenheit, below −20 degrees Fahrenheit, using cryogenic liquids such as liquid nitrogen at −273 degrees Fahrenheit. For example, the molds may be placed in a flash freezer at these low temperatures, which may also use convection to increase heat transfer. In a further embodiment, the molds may be directly exposed to a cryogenic liquid such as liquid nitrogen. In a further embodiment, the molds may be sized to have a narrow cross section to facilitate rapid freezing. For example, in one embodiment, the molds may be less than 1 inch in width. Narrow width mold embodiments also facilitate rapid freezing which prevents settling out or separation of important components, helping to ensure uniform or approximately uniform consistency. These smaller molds may be used in both conventional freezing systems as well as flash freezing embodiments at very low temperatures.

At 205, the frozen mixture may be demolded. For both large-scale and small-scale productions, the frozen mixtures may be briefly dipped into warm water or exposed to warm air to loosen them from the molds. In an industrial setting, the demolding process may be performed by automated machines. In a small-scale setting, the demolding process may be performed manually or using the automated system 306 controlled by the controller 303.

In some embodiments, after the demolding process, coatings, toppings, or drizzles may be added to the presently disclosed product to enhance its flavor and/or texture.

In some embodiments, after the demolding process, the presently disclosed product may be packaged and/or distributed.

Importantly, in many embodiments, an emulsifier and/or gelling agent may be crucial, if not critical, for maintaining the mixture's uniformity prior to and during the freezing process. As can be envisioned, in many cases various components may settle to a bottom of a vessel during the freezing process, which takes time. The denser, larger particle size and/or less hydrophilic component are more likely to settle to the bottom, resulting in a less uniform frozen composition. This, of course, poses a problem of non-uniform distribution of components in the frozen composition. The emulsifiers contemplated herein aid in keeping the components and particles thereof suspended in the mixture long enough for them to be held in place once frozen. Similarly, gelling agents thicken the mixture, especially as it cools, slowing settling and keeping components and particles suspended. In many embodiments, both gelling agents and emulsifiers may both be used. In many cases, in addition to aiding even distribution throughout the frozen composition, gelling agents provide a pleasant “soft” mouthfeel, improving product desirability. This is also true, though to a lesser extent, with many emulsifiers. Common emulsifier composition percentage by weight typically ranges between 0.1%-2%, but of course, this will vary depending on the emulsifier chosen, and suspended components and their tendency to settle. Common gelling agent composition percentage by weight typically ranges between 0.1%-2%, but of course this will vary depending on emulsifier chosen, and suspended components and their tendency to settle.

Examples of emulsifiers may include but are not limited to Acacia (gum Arabic), Acetic acid esters (ACETEM), Ammonium phosphatide, glycan, Brominated vegetable oil (BVO), Carboxymethylcellulose, cellulose, Caseinates, Carrageenan, DATEM (Diacetyl tartaric acid esters), Dextrin, guar gum, Lactic acid esters (LACTEM), Lecithin, Magnesium stearate, mono and diglycerides, phosphates, Polyglycerol esters (PGE), Pectin, Sodium Stearoyl Lactylate (SSL), Polysorbate, such as polysorbate 60, 65, 80, Propylene glycol esters of fatty acids (PGMS), Sodium stearolyllactylate (SSL), Ceteareth-20, Distilled Monoglycerides, Sorbitan Esters, Sucrose Acetate Isobutyrate, Sucrose fatty acid ester, Xantham Gum, and the like.

Examples of gelling agents may include but are not limited to Gelatin, Agar-Agar, Pectin, Carrageenan, Gellan Gum, Xanthan Gum, Guar Gum, Locust Bean Gum (Carob Gum), Psyllium Husk, Konjac Powder (Glucomannan), Sodium Alginate, Tara Gum, Agarose, Methylcellulose, Tamarind Gum, Poultry Collagen (Hydrolyzed Gelatin), and the like.

Furthermore, unless explicitly stated in the claims, the use of order, numbers, letters, or other names for processing elements and sequences are not intended to limit the order of the processes and methods of the present disclosure. While various examples have been discussed in the disclosure as currently considered useful embodiments of the invention, it should be understood that such details are provided for illustrative purposes only. The appended claims are not limited to the disclosed embodiments, and instead, the claims are intended to cover all modifications and equivalent combinations within the scope and essence of the embodiments disclosed in the present disclosure. For example, although the described system components may be implemented through a hardware device, they may also be realized solely through a software solution, such as installing the described system on an existing processing or mobile device.

Similarly, it should be noted that, for the sake of simplifying the presentation of embodiments disclosed in the present disclosure and aiding in understanding one or more embodiments of the present disclosure, various features have been sometimes combined into a single embodiment, drawing, or description. However, this manner of disclosure does not imply that the features required by the claims are more than the features mentioned in the claims. In fact, the features of the embodiments are less than all the features of the single embodiment disclosed in the foregoing disclosure.

In some embodiments, numeric values describing the composition and quantity of attributes are used in the description. It should be understood that such numeric values used for describing embodiments may be modified with qualifying terms such as “about,” “approximately” or “generally”. Unless otherwise stated, “about,” “approximately” or “generally” indicates that a variation of ±20% is permitted in the described numbers. Accordingly, in some embodiments, the numerical parameters used in the disclosure and claims are approximations, which can change depending on the desired characteristics of the individual embodiment. In some embodiments, the numerical parameters should take into account a specified number of valid digits and employ a general manner of bit retention. Although the numerical ranges and parameters used in some embodiments of the present disclosure to confirm the breadth of the range are approximations, in specific embodiments, such numerical values are set as precisely as practicable.

With respect to each of the patents, patent applications, publications of patent applications, and other material, such as articles, books, specifications, publications, documents and the like, cited in the present disclosure, the entire contents thereof are hereby incorporated herein by reference. Application history documents that are inconsistent with the contents of the present disclosure or that create conflicts are excluded, as are documents (currently or hereafter appended to the present disclosure) that limit the broadest scope of the claims of the present disclosure. It should be noted that in the event of any inconsistency or conflict between the descriptions, definitions, and/or use of terminology in the materials appended to the present disclosure and the contents described herein, the descriptions, definitions, and/or use of terminology in the present disclosure shall prevail.

In closing, it should be understood that the embodiments described in the present disclosure are used only to illustrate the principles of the embodiments of the present disclosure. Other deformations may also fall within the scope of the present disclosure. Therefore, by way of example and not limitation, alternative configurations of the embodiments disclosed in the present disclosure may be considered consistent with the teachings of the present disclosure. Accordingly, the embodiments described in the present disclosure are not limited to the explicitly introduced and described embodiments in the present disclosure.

What is claimed is:

1. A product, comprising:
a mixture, including:
one or more nutraceuticals or pharmaceuticals;
one or more vegetable, herb, fungus, or fruit extracts;
a base that is liquid at room temperature;
an emulsifier or gelling agent;
wherein, the mixture is frozen in one or more molds.

2. The product of claim 1, wherein the base comprises electrolytes.

3. The product of claim 1, wherein the one or more nutraceuticals or pharmaceuticals are in liquid or powdered forms.

4. The product of claim 1, wherein the mixture further includes food additives.

5. The product of claim 1, wherein a stick, grip, or handle is inserted into each of the one or more molds.

6. The product of claim 1,
wherein the one or more nutraceuticals or pharmaceuticals include melatonin, L-theanine, GABA, and other amino acids;
wherein the one or more vegetable, herb, fungus, or fruit extracts include chamomile extracts;
wherein the mixture further includes date sugar, natural flavorings, fruit juices, and essential oil;
wherein the liquid base is filtered water or comprises electrolytes.

7. The product of claim 1,
wherein the one or more nutraceuticals or pharmaceuticals include Vitamin C, GABA, and zinc;
wherein the one or more vegetable, herb, fungus, or fruit extracts include clove extracts, chamomile extracts, and elderberry extracts;
wherein the mixture further includes Manuka honey;
wherein the liquid base comprises electrolytes.

8. The product of claim 1,
wherein the one or more nutraceuticals or pharmaceuticals include GABA;
wherein the one or more vegetable, herb, fungus, or fruit extracts include chamomile extracts and passion fruit extracts;
wherein the mixture further includes date juice;
wherein the liquid base comprises electrolytes.

9. The product of claim 1,
wherein the one or more nutraceuticals or pharmaceuticals include Vitamin B6;
wherein the one or more vegetable, herb, fungus, or fruit extracts include fennel extracts and roman chamomile extracts;
wherein the mixture further includes natural flavor enhancers or Manuka honey;
wherein the liquid base comprises electrolytes.

10. The product of claim 1,
wherein the one or more nutraceuticals or pharmaceuticals include Vitamin B6;
wherein the one or more vegetable, herb, fungus, or fruit extracts include ginger extracts and mint extracts;
wherein the mixture further includes protein-based additives;
wherein the liquid base comprises juice.

11. A method for preparing a product, comprising:
selecting one or more nutraceuticals or pharmaceuticals, one or more vegetable, herb, fungus, or fruit extracts, and a base that is liquid at room temperature;
preparing a mixture of the one or more nutraceuticals or pharmaceuticals, the one or more vegetable, herb, fungus, or fruit extracts, and the base;
adding an emulsifier or gelling agent to the mixture;
pouring the mixture into one or more molds;
freezing the one or more molds containing the mixture;
demolding the mixture from the one or more molds.

12. The method of claim 11, wherein the selection of the one or more nutraceuticals or pharmaceuticals, the one or more vegetable, herb, fungus, or fruit extracts, and the base is performed electronically via an I/O device, connected to a controller.

13. The method of claim 11, wherein a plurality of dispensers, each containing a health-enhancing substance from the one or more nutraceuticals or pharmaceuticals and the one or more vegetable, herb, fungus, or fruit extracts, is operable to automatically dispense the health-enhancing substance, based on the selection of the one or more nutraceuticals or pharmaceuticals and the one or more vegetable, herb, fungus, or fruit extracts.

14. The method of claim 13, wherein the plurality of dispensers is connected to a controller.

15. The method of claim 11, wherein the one or more molds containing the mixture are frozen in a freezer.

16. The method of claim 11, wherein the one or more molds containing the mixture are frozen by rapid freezing.

17. The method of claim 11, wherein the base includes filtered water, electrolytes, juice, milk, or plant-based milk.

18. The method of claim 11, wherein a stick, grip, or handle is inserted into the one or more molds.

19. The method of claim 11, wherein the one or more nutraceuticals or pharmaceuticals are in liquid or powdered form.

20. The method of claim 11, further comprising:
selecting one or more food additives;
adding the one or more food additives to the mixture.
